(19) United States
(12) Patent Application Publication
(10) Pub. No.: US 2005/0165006 A1
(43) Pub. Date: Jul. 28, 2005

(54) Quinoline derivatives, their preparation and pharmaceutical compositions comprising the same

(76) Inventors: Dae-Yoon Chi, Seoul (KR); Byoung-Se Lee, Manan-ku Anyang-si Kyunggi-do (KR); Soyoung Chu, Nam-ku Incheon (KR); Byung-Chul Lee, Seoul (KR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/509,483

(22) PCT Filed: Apr. 3, 2002
(86) PCT No.: PCT/KR02/00595

(51) Int. Cl. A61K 31/551; A61K 31/496; C07D 43/02; A61K 31/47; C07D 215/38

(52) U.S. Cl. 514/218; 514/253.06; 514/313; 540/575; 544/363; 546/159

(57) ABSTRACT

Novel quinoline derivatives were prepared and evaluated their pharmaceutical activities. The quinoline derivatives according to the present invention effectively bind serotonin transporter (SERT) which is called serotonin reuptake site. Serotonin is one of the neurotransmitter and the lack of its concentration in synapse cause the depression. The quinoline derivatives in this invention can interrupt reuptake of serotonin into presynaptic neuron resulting the increasement of concentration of serotonin in synapse as well as stimulating the signal through the binding with serotonin recepter. Thus, they can be used for the prevention and treatment of mental disorder, especially depression, caused by the deficiency of serotonin concentration in synapse.

### TECHNICAL FIELD

The present invention relates to quinoline derivatives represented by in formula 1 as below. More specifically, the present invention relates to quinoline derivatives and their pharmaceutically acceptable salts that interrupt the reuptake of serotonin into presynaptic neuron and thus increase the concentration of serotonin in synapse. The present invention also includes the process for preparing the said compounds of formula 1 and their pharmaceutical compositions to prevent or treat serotonin-related mental disorders comprising the said compounds as effective ingredients.

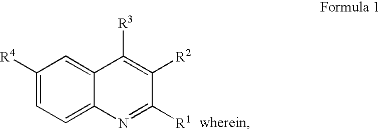

wherein,
R¹ is piperazinyl, 2-methylpiperazinyl, diazepinyl or N-methyl-N-(2-N′-methylamino)ethylamine;
R² is H, halogen atom, C₁˜C₄ alkyl or C₁˜C₄ haloalkyl;
R³ is H, halogen atom, vinyl or furanyl group; and
R⁴ is halogen atom or nitro group.

### BACKGROUND ART

Mental disorders, regarded as advanced country type diseases, are increasing significantly according to the rapid growth of society and the advance of civilization, and it pointed out to be a heavy burden of stress as a major factor of onset although it acts a little on a congenital cause.

Because mental disorder also does not reveal an obvious symptom as depression, it is very difficult to catch the exact diagnosis. So far, the symptoms are estimated by having the interview with a psychiatrist. Those who show mental symptoms such as loss of desire, lack of worth, thinking subsequently about death, and attempted suicide; and physical symptoms such as anorexia dyspepsia, sleep disorder, and loss of weight are judged to have fall into depression. In general, it is known that most women have an attack of depression and 10˜25% of women have the probability to onset depression in a lifetime whereas men get only 5˜12%. In addition, it is estimated that 5˜9% of woman and 2˜3% of men are indeed melancholiacs (patients undergoing depression).

Mental activity is operated by neurotransmitters subsequently acting in the human brain. For normal consideration, the concentration of these neurotransmitters should be maintained in a certain level at the brain. The variation of the concentration such as the increase or decrease of the concentration causes mental disorder. Even though research on the relationship between neurotransmitters and mental disease caused by trouble in the neurotransmitters is continuously conducted, it is regarded that subsequent stress causes the change of the brain activity, its factor of onset and action mechanism have not been found out yet.

Meanwhile, serotonin, a member of neurotransmitter acting in the brain, is known to be a material related to mental disorder, especially, an immediate cause of depression.

The action mechanism of serotonin is like that of other neurotransmitters. Concretely, the serotonin is biosynthesized from tryptophan, a member of amino acid, by enzyme in neurons. The biosynthesized serotonin is secreted at the presynaptic neuron terminus surrounded with endo cytoplasma reticula. Thus the secreted serotonin binds with the serotonin receptor located at the cell membrane of postsynaptic neuron via synapse, subsequently, transfers a signal to the next neuron. After finishing its role, the serotonin is dissociated from receptor, it is transported via serotonin transporter (SERT) which is located at the presynaptic neuron and regarded as a reuptake site. As it is known that depression is caused by the defection of serotonin concentration in synapse, it is preferably possible to maintain the concentration of serotonin in synapse when a drug that powerfully and selectively inhibits SERT is administrated.

Our country has little understanding of depression, and has a low-pitched interest in developing an depressant agent. However, it is reported that approximately the million patients undergo depression treatment in USA every year, thus, the total expenses estimated by decreasing of productivity, reduction of, cost for treatment and regeneration are over approximately 40 trillion dollars. Among thus the cost for depression treatment takes up over 4 trillion dollars. For example, Fluoxetine, a product of Lilly in USA, offers a sale of 1.7 trillion dollars every year regardless of its particular side effects such as emesis, pruritus, and retardation of drug action.

As for the selective SERT inhibitor classified into the second-generation anti-depression agent, besides Fluoxetine, Paroxetin and Setrallin have already been commercialized. With Fluoxetin (Ki=16 nM), Paroxetine (Ki=0.24 nM), and Setrallin (Ki=0.39 nM), 6-nitro-2-(piperazin-1-yl)quinoline (6-nitroquipazine, Ki=0.018 nM) is known to be a compound having more powerful binding affinity (Ki) to SERT. However, there is little report on the structure and activity relationships as well as the synthesis of its derivatives.

Leading to the present invention, conducted by the present inventors aiming to develop a novel selective SERT inhibitor having an excellent binding affinity and high selectivity using 6-nitro-2-(piperazin-1-yl) quinoline and its derivatives, resulted in finding that quinoline derivatives represented by formula 1 have equal or stronger binding affinity to SERT in comparison with that of 6-nitro-2-(piperazin-1-yl)quinoline, as known in this art.

### SUMMARY OF THE INVENTION

It is an object of the present invention to provide compounds, with a high selectivity and an excellent binding affinity to SERT, as a third-generation anti-depression agent being able to solve problems of that of the second-generation.

It is another object of the present invention to provide compounds with a high selectivity and an excellent binding affinity to SERT.

### BRIEF DESCRIPTION OF THE DRAWINGS

In the drawings:

FIG. 1 is a graph showing the anti-depression effect evaluated by a forced swimming test when 50 mg/kg of 4-chloro-6-nitro-2-(piperazin-1-yl)quinoline is administered to depression-induced mice;

FIG. 2A is a graph illustrating the anti-depression effect obtained by measuring immobility time using a tail suspension test wherein 50 mg/kg or 5 mg/kg of 4-chloro-6-nitro-2-(piperazin-1-yl)quinoline is administered to depression-induced mice and 0.5% dimethylsulfoxide (DMSO) is administered to them as a control;

FIG. 2B is a graph illustrating the anti-depression effect obtained by measuring of immobility time using a tail suspension test wherein 1 mg/kg or 10 mg/kg of 4-chloro-6-nitro-2-piperazine is administered to depression-induced mice and 0.5% dimethylsulfoxide (DMSO) is administered to them as a control.

### DISCLOSURE OF INVENTION

The present invention provides quinoline derivatives and their pharmaceutical acceptable salt represented by the following formula 1: 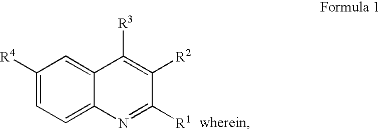
R¹ is piperazinyl, 2-methylpiperazinyl, diazepinyl or N-methyl-N-(2-N′-methylamino)ethylamine group;
R² is H, halogen atom, C₁˜C₄ alkyl or C₁˜C₄ haloalkyl;
R³ is H, halogen atom, vinyl or furanyl group; and
R⁴ is halogen atom or nitro group.

More preferably,
R¹ is 2-methylpiperazinyl, diazepinyl or N-methyl-N-(2-N′-methylamino)ethylamine group;
R² is H, bromine, methyl, ethyl, propyl, chloropropyl or fluoropropyl group;
R³ is H, chorine, bromine, iodine, vinyl or 2-furanyl group; and
R⁴ is chlorine, bromine or nitro group.

Most preferably, examples of the compounds represented by the chemical formula 1 include the following table 1:

TABLE 1

| Example | Compound | Formula |
|---|---|---|
| 1 | 3-methyl-6-nitro-2-(piperazin-1-yl)quinoline | 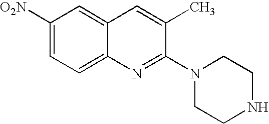 |
| 2 | 3-ethyl-6-nitro-2-(piperazin-1-yl)quinoline | 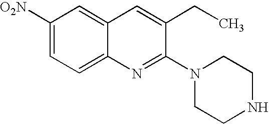 |
| 3 | 6-nitro-2-(piperazin-1-yl)-3-propylquinoline | 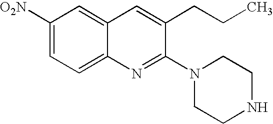 |
| 4 | 3-(3-chloropropyl)-6-nitro-(piperazin-1-yl)quinoline | 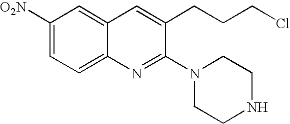 |
| 5 | 6-iodo-2-(piperazin-1-yl)quinoline | 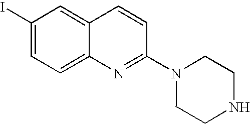 |
| 6 | 6-bromo-2-(piperazin-1-yl)quinoline | 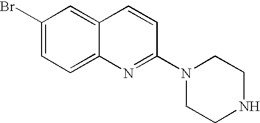 |
| 7 | 6-chloro-2-(piperazin-1-yl)quinoline | 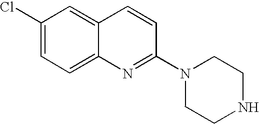 |
| 8 | 3-(3-fluoropropyl)-6-nitro-2-(piperazin-1-yl)quinoline | 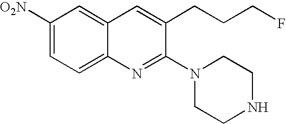 |
| 9 | 3-bromo-6-nitro-2-(piperazin-1-yl)quinoline | 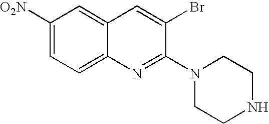 |
| 10 | 4-chloro-6-nitro-2-(piperazin-1-yl)quinoline | 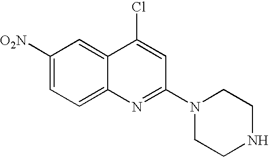 |
| 11 | 4-bromo-6-nitro-2-(piperazin-1-yl)quinoline | 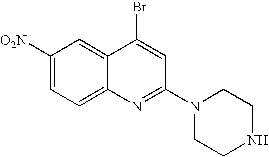 |
| 12 | 4-iodo-6-nitro-2-(piperazin-1-yl)quinoline | 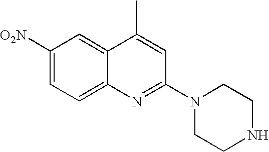 |
| 13 | 6-nitro-2-(piperazin-1-yl)-4-vinylquinoline | 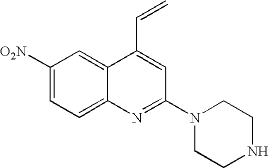 |
| 14 | 4-(2-furanyl)-6-nitro-2-(piperazin-1-yl)quinoline | 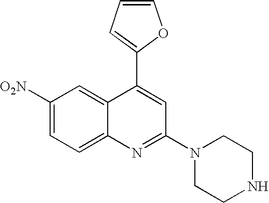 |
| 15 | 2-(3-methylpiperazin-1-yl)-6-nitroquinoline | 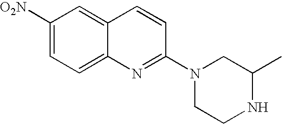 |
| 16 | 2-(N-methyl-N-(2-N′-methylamino)ethyl)amino-6-nitroquinoline | 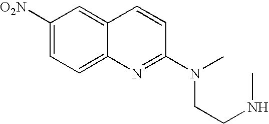 |
| 17 | 2-[1,4]diazepin-1-yl-6-nitroquinoline | 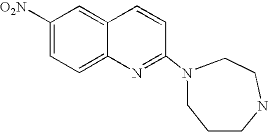 |

The present invention further includes solvated compounds and hydrates prepared by use of the quinoline derivatives of formula 1 and their pharmaceutically acceptable salts.

The compounds of formula 1 in accordance with the present invention may be utilized in the form of pharmaceutically acceptable salts and the acid addition salts prepared by adding pharmaceutically acceptable free acids are useful. Compounds of formula 1 may be changed to the corresponding acid addition salts according to the general practices in this field. Both inorganic and organic acids may be used as free acids in this case. Examples of the inorganic free acid include hydrochloric acid, phosphoric acid, sulfuric acid and nitric acid. Available organic free acids are exemplified by methanesulfonic acid, p-toluene sulfonic acid, acetic acid, trifluoroacetic acid, citric acid, maleic acid, succinic acid, oxalic acid, benzoic acid, tartaric acid, fumaric acid, manderic acid, propionic acid, lactic acid, glycolic acid, gluconic acid, galacturonic acid, glutamic acid, glutaric acid, glucuronic acid, aspartic acid, ascorbic acid, carbonic acid, vinylic acid, and iodoic acid.

The acid addition salts of the compounds according to the present invention are prepared in a customary manner, for example by dissolving the compounds of formula 1 in excess aqueous acid and precipitating the salt with a water-miscible organic solvent, such as methanol, ethanol, acetone or acetonitrile.

It is also possible to prepare the compounds by heating equivalent amounts of the compounds of formula 1 and acid in water or an alcohol, such as glycol monomethyl ether, and then drying the mixture or suction filtering off the precipitated salts.

Further, the acid addition salts of the compounds according to the present invention are prepared by dissolving the compounds of formula 1 in organic solvents such as diethylether, tetrahydrofuran and dichloromethane, acetonitrile, precipitating the salts obtained by adding organic acid and inorganic acid, as described above, and can be, followed by suction filtering.

Further, the present invention provides a method for preparing the quinoline derivative of formula 1.

In accordance with another aspect of the present invention, there is provided a method for preparing an 2-piperazin-1-yl-quinoline, represented by the following reaction scheme 1:

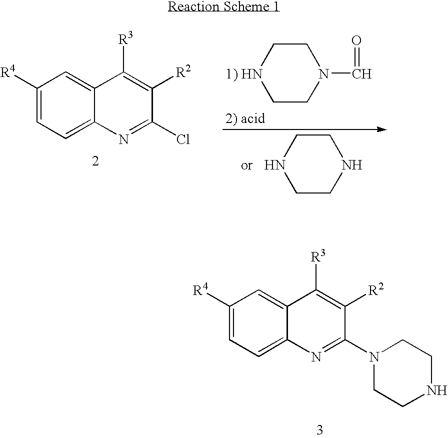

wherein,
R² is H, bromine, methyl, ethyl, propyl, chloropropyl or fluoropropyl group;
R³ is H, chlorine or bromine group; and
R⁴ is chlorine, bromine or nitro group.

As shown in the above reaction scheme 1, the preparation of 2-piperazin-1-yl-quinolines of formula 3 is achieved by reacting the quinoline compounds of formula 2 with 1-piperazinecarboxaldehyde, and treating thus obtained mixture with acid compounds to introduce piperazinyl group at 2-position of quinoline compounds of formula 2.

Preferably, the substitution of piperazinyl group is conducted from 20° C. to 140° C. More preferably, when 1-piperazinecarboxaldehyde is used as a reagent, the reaction is carried out from 20° C. to 80° C. before dissolving in acid solution such as sulfuric acid to remove an aldehyde compound produced by the substitution reaction.

In accordance with another aspect of the present invention, there is provided a method for preparing an 4-iodo-6-nitro-2-piperazin-1-yl-quinoline, represented by the following reaction scheme 2:

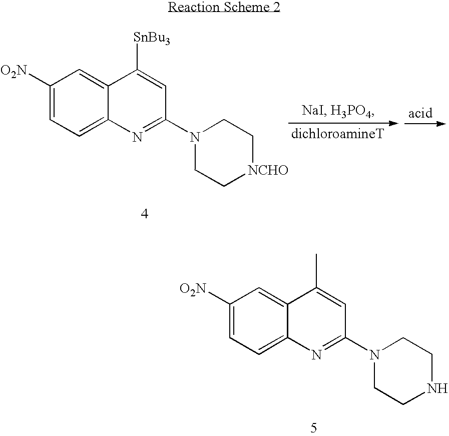

As shown, the 4-iodo-6-nitro-2-piperazin-1-yl-quinoline of formula 5 is prepared by:
a) reacting 4-tributylstannyl-6-nitro-2-(4-formylpiperazin-1-yl)quinoline of formula 4 with NaI in the presence of phosphoric acid and dichloroamine T to introduce iodine at 4-position of compounds of formula 4; and
b) treating thus obtained mixture with acid compound.

In accordance with still another aspect of the present invention, there is provided a method for preparing an 6-nitro-2-piperazin-1-yl-4-vinylquinoline and 4-(2-furanyl)-6-nitro-2-piperazin-1-yl-quinoline, represented by the following reaction scheme 3:

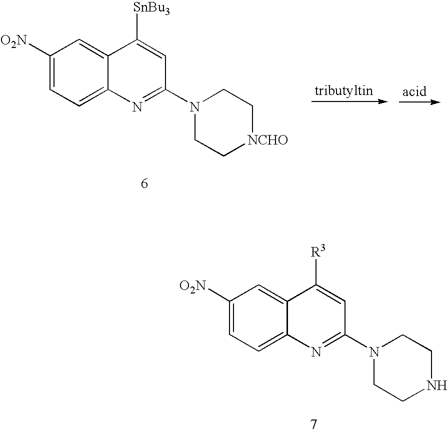

As shown, the 6-nitro-2-piperazin-1-yl-4-vinylquinoline and 4-(2-furanyl)-6-nitro-2-piperazin-1-yl-quinoline are prepared by:
a) Stille coupling reactions of 4-bromo-6-nitro-2-(4-formylpiperazine-1-yl)-quinoline with tributyl(vinyl)tin or tributyl(2-furanyl)tin in the presence of palladium catalyst to introduce vinyl or furanyl at 4-position of compounds of formula 6; and
b) treating thus obtained mixture with acid compounds.

Preferably, the Stille reaction is carried out from 90° C. to 120° C. under inert gas, e.g., N₂(g).

In this case, it is possible to prepare the compounds substituted with aryl, heteroaryl and aryl group besides vinyl and furanyl group.

In accordance with yet another aspect of the present invention, there is provided a method for preparing an 2-(3-methylpiperazin-1-yl)-6-nitroquinoline, 2-(N-methyl-N-(2-N′-methylamino)ethylamino-6-nitroquinoline and 2-[1,4]diazepin-1-yl-6-nitroquinoline, represented by the following reaction scheme 4:

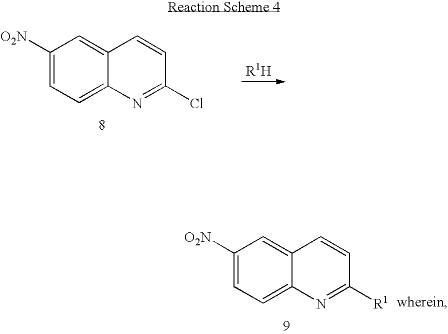

R¹ is 2-methylpiperazinyl, N-methyl-N-(2-N′-methylamino)ethylamine or diazepinyl group.

As shown, the compounds of formula 9 including an 2-(3-methylpiperazin-1-yl)-6-nitroquinoline, 2-(N-methyl-N-(2-N′-methylamino)ethylamino-6-nitroquinoline and 2-[1,4)diazepin-1-yl-6-nitroquinoline is prepared by reacting 2-chloro-6-nitroquinoline of formula 8 with 2-methylpiperazine, N-methyl-N-(2-N′-methylamino)ethylamine or diazepine.

In accordance with a further aspect of the present invention, there is provided a pharmaceutical composition comprising the compounds of formula 1 as an effective ingredient to prevent or treat mental disorder caused by serotonin.

Concretely, the quinoline derivatives of the invention may be utilized to prevent or treat mental disorders, especially to depression.

In a preferable embodiment of the present invention, the brain tissue was gently isolated from a mouse, grounded, and then the biological activity against SERT was measured in a variety of concentration. As a result, the compounds of the present invention shows a much higher Ki value than that of the already commercialized Fluoxetine and a similar value to that of Paroxetine. As shown table 3, especially, 4-chloro-6-nitro-2-piperazin-1-yl-quinoline shows an excellent binding affinity over ten times than 6-nitro-2-piperazin-1-yl-quinoline.

Further, the compounds of the present invention show an excellent anti-depression activity. In an embodiment of the invention, a forced swimming test was performed to the mouse injected with the compounds of the invention. As a result, it is found that the compounds of the invention show high anti-depression activity in comparison with the control. In addition, the same result is observed in tail suspension test.

The compounds of formula 1 in accordance with the present invention can be formulated into various dosage forms for oral or parenteral administration. For formulation, pharmaceutically acceptable fillers, thickeners, binders, wetting agents, disintergrants, surfactants or expedients may be used.

Tablets, pills, powders, granules, and capsules exemplify solid dosage forms for oral administration. These solid forms are prepared by admixing at least one compound of the chemical formula 1 with at least one expedient, such as starch, calcium carbonate, sucrose, lactose, gelatin, etc. In addition to expedients, a lubricant such as magnesium stearate may be added.

Exemplified by suspensions, internal solutions, emulsions, syrups, etc., liquid dosage forms for oral administration may comprise simple diluents, such as water and liquid paraffin, as well as wetting agents, sweeteners, aromatics, and/or perspectives.

Dosage forms for parenteral administration include sterile aqueous solutions, non-aqueous solvents, suspensions, emulsions, freeze-dried agents, suppositories, etc. For formulation of non-aqueous solvents and suspensions, vegetable oils, such as propylene glycol and polyethylene glycol, or injectable esters such as ethyl oleate, may be used. As bases for suppositories, Witepsol, macrogol, Tween 61, cocoa butter, laurinic acid, and glycerogelatine are useful.

In general, the compounds of the formula 1 may be administered in a total dose of 0.01˜500 mg, more preferably 0.1˜20 mg, to adults in 1 or several installments a day. However, the dose may vary depending on the conditions of the subject, including, for example, physical constitutions and weights of patients, kinds and severity of diseases, administration routes and intervals, etc.

A better understanding of the present invention may be obtained in light of the following examples which are set forth to illustrate, but are not to be construed to limit the present invention.

### PREPARATION EXAMPLE 1

### Preparation of 2-chloro-3-methyl-6-nitroquinoline

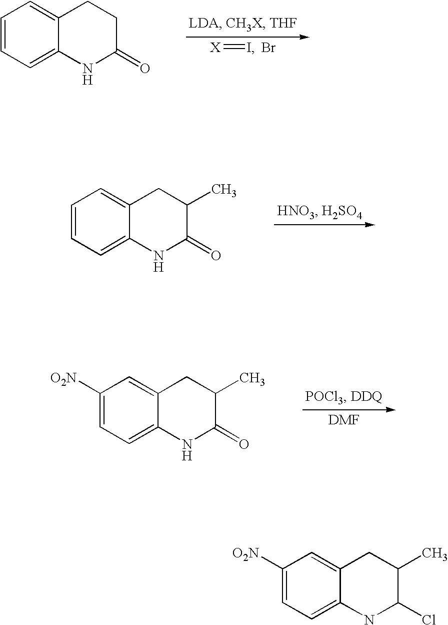

### Step 1) Preparation of 3-methylhydrocarbostiril

In 15 ml of anhydrous tetrahydrofuran, hydrocarbostyril (1.00 g, 6.79 mmol) was dissolved, the mixture was added 2 M LDA(lithium diisopropylamide, 7.47 ml, 14.94 mmol) and then stirred at −78° C. for 1 hour. The obtained mixture was slowly added iodomethane (0.44 ml, 7.13 mmole) at the same temperature and then reacted for 1 hour by slowly heating it up to room temperature. The desired product was obtained by column chromatography of thus obtained mixture.

### Step 2) Preparation of 3-methyl-6-nitro-hydrocarbostyril

In 10 ml of conc. H₂SO_{4,} the compound (586 mg, 3.64 mmol) prepared in the step 1) was dissolved, the mixture was slowly added conc. H₂SO₄ (0.30 ml, 4.00 mmol), and then stirred for 5 minutes. After the completion of stirring, the mixture was carefully poured into ice water, concentrated, and then dissolved in ethyl acetate. The desired product was obtained by column chromatography of thus obtained mixture.

### Step 3) Preparation of 2-chloro-3-methyl-6-nitroquinoline

In 15 ml of N,N′-dimethylformamide, the compound (586 mg, 2.84 mmol) prepared in the step 2) and DDQ (2,3-dichloro-5,6-dicyanobenzoquinone, 1.1 equivalent, 724 mg, 3.13 mmol) as an oxidant were dissolved, the mixture was added POCl₃ (phosphorous oxychloride, 0.41 ml, 4.26 mmol) to react at room temperature for 1 hour. The obtained mixture was carefully poured into ice water to produce a precipitate. The precipitate was filtered under reduced pressure and then dissolved in ethylacetate. The desired product was obtained by column chromatography of the obtained mixture.

### PREPARATION EXAMPLE 2˜5

The desired products including 2-chloro-3-ethyl-6-nitroquinoline, 2-chloro-6-nitro-3-propylquinoline, 2-chloro-3-(3-chloropropyl)-6-nitroquinoline or 2-chloro-3-(3-chloropropyl)-6-nitroquinoline were prepared as the same manner in preparation example 1, except for using iodomethane, 1-chloro-3-iodopropane, 1-bromo-3-fluoropropane or hydroiodide instead of iodomethane as a starting material.

### PREPARATION EXAMPLE 6

### Preparation of 2-chloro-6-iodoquinoline

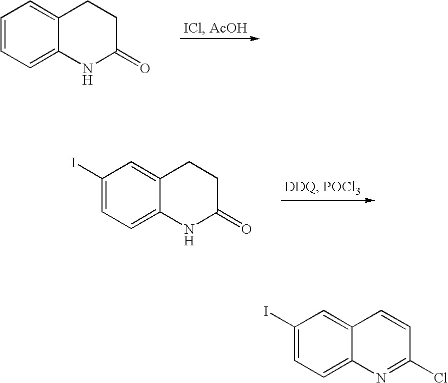

### Step 1) Preparation of 6-iodocarbostyril

In 10 ml of acetic acid, hydrocarbostyril (1.00 g, 6.79 mmol) was dissolved, and slowly added ICI (iodine monochloride, 1.1 equivalent, 0.40 ml) at room temperature to react 0.5 hours with stirring. After the completion of the reaction, thus obtained mixture was poured into ice water to produce a precipitate. The precipitate was filtered under reduced pressure and then dissolved in ethylacetate. The desired product was obtained by column chromatography of the obtained mixture.

### Step 2) Preparation of 2-chloro-6-iodoquinoline

The desired product was prepared by chlorination as described in step 3 of the Preparation Example 1.

### PREPARATION EXAMPLE 7

### Preparation of 6-bromo-2-chloroquinoline

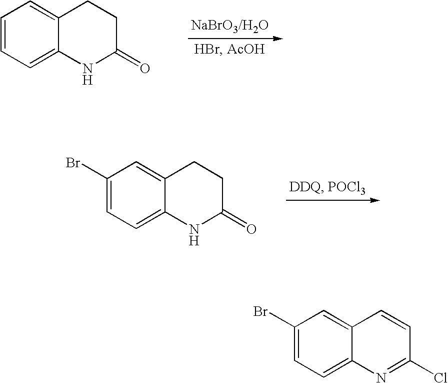

### Step 1) Preparation of 6-bromohydrocarbostyril

In a mixture of acetic acid (40 ml) and bromic acid (10 ml), hydrocarbostyril (1.00 g, 6.79 mmol) was dissolved, added NaBrO₃ (366 mg, 2.38 mmol) dissolved in 2 ml of water, and then stirred at room temperature for 0.5 hours. The obtained mixture was carefully poured into ice water to produce a precipitate. The precipitate was filtered under reduced pressure and then dissolved in ethylacetate. The desired product was obtained by column chromatography of the obtained mixture.

### Step 2) preparation of 6-bromo-2-chloroquinoline

The desired product was prepared by chlorination as described in the step 3 of the Preparation Example 1.

### PREPARATION EXAMPLE 8

### Preparation of 2,6-dichloroquinoline

The desired product was prepared as the same manner in the Preparation Example 7, except for using hydrochloric acid and NaClO₃ instead of bromic acid and NaBrO₃, respectively.

### PREPARATION EXAMPLE 9

### Preparation of 3-bromo-2-chloro-6-nitroquinoline

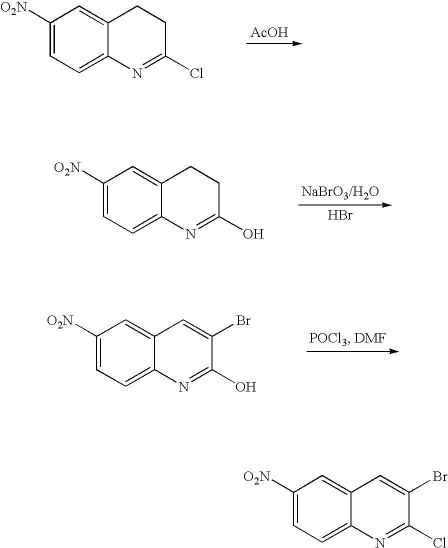

### Step 1) Preparation of 2-hydroxyl-6-nitroquinoline

In 90 ml of acetic acid, 2-chloro-6-nitroquinoline (2.22 g, 10.65 mmol) was dissolved, and then was refluxed for 10 hours to react. After the completion of reaction, the obtained mixture was cooled to room temperature and poured into ice water to produce a precipitate. The desired product was obtained by washing the precipitate with water several times followed by drying.

### Step 2) Preparation of 3-bromo-2-hydroxyl-6-nitroquinoline

In 50 ml of 48% bromic acid, the compound prepared in the step 1) were dissolved, the mixture was added 10 ml of NaBrO₃ aqueous solution (790 mg, 5.23 mmol), and then was stirred at 100° C. for 3 hours. After the completion of stirring, the mixture was poured into ice water to produce a precipitate, filtered under reduced pressure, and then dried. After dissolved in ethylacetate, the desired product was obtained by column chromatography of thus obtained mixture.

### Step 3) Preparation of 3-bromo-2-chloro-6-nitroquinoline

In 50 ml of DMF, the compound (619 mg, 2.30 mmol) prepared in the step 2) was dissolved, the mixture was slowly added 0.66 ml of POCl₃ (3 equivalent), and then was stirred at room temperature for 1 hour. After the completion of stirring, the mixture was poured into ice water to produce a precipitate, filtered under reduced pressure, and then dissolved in ethylacetate. The desired product was obtained by column chromatography of the obtained mixture.

### PREPARATION EXAMPLE 10

### Preparation of 2,4-dichloro-6-nitroquinoline

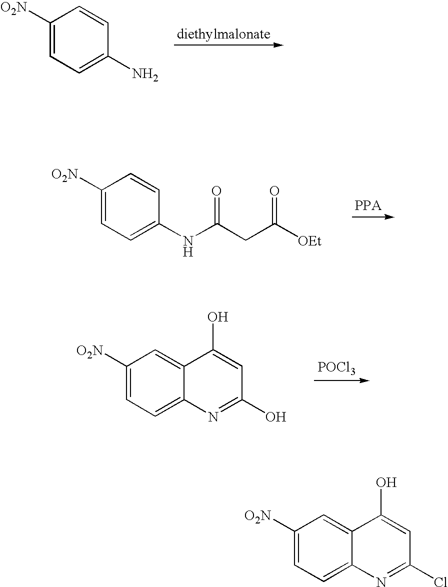

### Step 1) Preparation of 4-nitroaniline-N-ethylmalonate

In 44 ml of diethylmalonate (289.60 mmol), 4-nitroaniline (10.00 g, 72.40 mmol) was dissolved, and then reacted at 150° C. for 6 hours. The obtained mixture was extracted with ethylacetate and then condensed under vacuum. The desired product was obtained by column chromatography of the obtained residue.

### Step 2) Preparation of 2,4-dihydroxy-6-nitroquinoline

In 100 g of polyphosphoric acid (PPA), the compound prepared in the step 1) was dissolved, and then reacted at 110° C. for 1 hour. The obtained mixture was slowly poured into ice water to produce a precipitate. The solid was filtered under reduced pressure and then dissolved in ethylacetate. The desired product was obtained by column chromatography of the mixture.

### Step 3) Preparation of 2,4-dichloro-6-nitroquinoline

In 40 ml of POCl₃, without solvent, the compound (2.00 g, 9.70 mmol) prepared in the step 2) was dissolved, and then stirred at room temperature for 1 hour. After POCl₃ was evaporating under vacuum, thus obtained mixture was added water, extracted with ethylacetate, and then concentrated under reduced pressure. The desired product was obtained by column chromatography of thus obtained residue.

### PREPARATION EXAMPLE 11

### Preparation of 2,4-dibromo-6-nitroquinoline

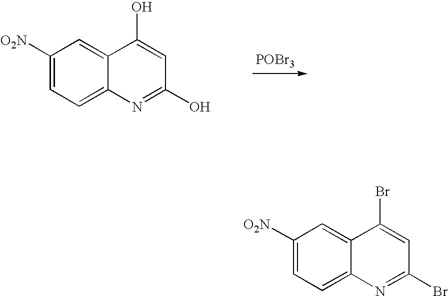

In 80 ml of 1,4-dioxane, 2,4-dihydroxy-6-nitroquinoline (4.00 g, 19.40 mmol) prepared in step 2 of the Preparation Example 10 was dissolved, slowly added POBr₃ (16.69 g, 58.21 mmol), and the refluxed for 6 hours to react them. After the completion of the reaction, the mixture was poured into ice water, extracted with ethylacetate, and then concentrated under reduced pressure. The desired product was obtained by column chromatography of thus obtained residue.

### PREPARATION EXAMPLE 12

### Preparation of 4-tributylstannyl-6-nitro-2-(4-formylpiperazin-1-yl)quinoline

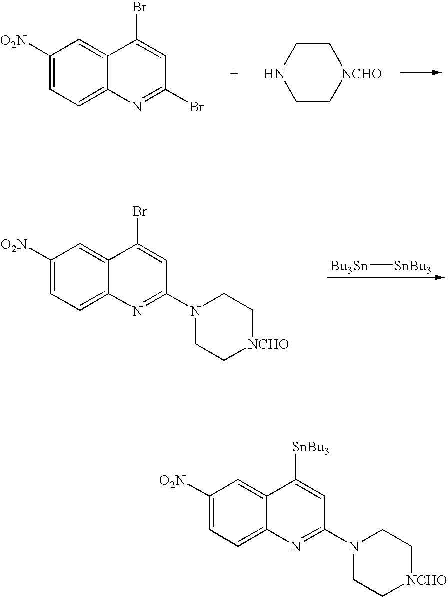

### Step 1) Preparation of 4-bromo-6-nitro-2-(4-formylpiperazin-1-yl)quinoline

In 100 ml of DMF, 2,4-dibromo-6-nitroquinoline (7.33 g, 22.08 mmol) was dissolved, added 1-piperazinecarboxaldehyde (6.70 ml, 55.20 mmol), and the stirred at room temperature for 1 hour. After pouring the mixture into ice water, the desired product was isolated by filtration under vacuum.

### Step 2) Preparation 4-tributylstannyl-6-nitro-2-(4-formylpiperazin-1-yl)quinoline

In 60 ml of dioxane, the compound (3 g, 8.22 mmol) prepared in step 1) and tetrakis(triphenylphosphin)palladium (0) were dissolved under inert gas, added bistributyltin (4.81 ml, 9.04 mmol) at room temperature, and then refluxed for 3 hours. After the completion of the reaction, the mixture was cooled to at room temperature and passed through a column filled with celite. Thus obtained mixture was added water and ammonia water, respectively, extracted with dichloromethane to provide the desired product.

### PREPARATION EXAMPLE 13

### Preparation of 2-chloro-6-nitroquinoline

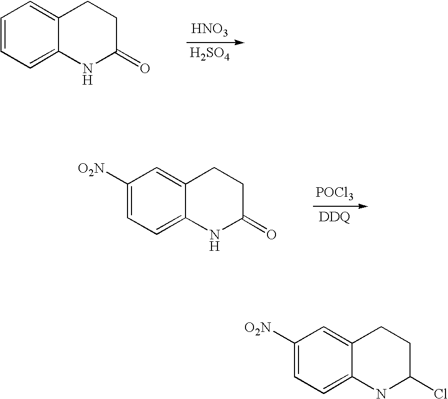

### Step 1) Preparation of 6-nitrohydrocarbostyril

In 20 ml of conc. H₂SO₄, hydrocarbostyril (1.00 g, 6.79 mmol) was dissolved, added 0.56 ml of conc. HNO₃ (1.1 equivalent), and then stirred at room temperature for 10 minutes. After the completion of stirring, the mixture was slowly poured into ice water to produce a precipitate, filtered under reduced pressure, dried, and then thus obtained precipitate dissolved in ethylacetate. The desired product was obtained by column chromatography of the obtained mixture.

### Step 2) Preparation of 2-chloro-6-nitroquinoline

The desired product was prepared by chlorination as described in step 3 of the-example preparation 1.

### EXAMPLE 1

### Preparation of 3-methyl-6-nitro-2-piperazin-1-yl-quinoline

### Step 1) Preparation of 3-methyl-6-nitro-2-(4-formylpiperazin-1-yl)quinoline

In 30 ml of DMF, 2-chloro-3-methyl-6-nitroquinoline (609 mg, 2.74 mmol) prepared in the Preparation Example 1 was dissolved, added 1-piperazinecarboxaldehyde (1.00 ml, 8.21 mmol) at room temperature, and then stirred at 100 ° C. for 3 hours. The mixture was cooled, added water to produce a precipitate, washed with 50 ml of water, and then dried over for 1 hour in vacuo to give the precipitate.

### Step 2) Preparation of 3-methyl-6-nitro-2-piperazin-1-yl-quinoline

In 30 ml of 4 M conc. H₂SO₄ aqueous solution, the product prepared in step 1) was dissolved and stirred at 80° C. for 3 hours. To the mixture was added 50 ml of water and further added 4 N NaOH aqueous solution to adjust pH=12. Thus obtained precipitate was isolated by filtration, washed with 50 ml of water, and then dried for 1 hour in vacuo over to give a yellowish precipitate (402 mg, 1.51 mmol, 55%).

mp 164-166° C.;

¹H NMR (200 MHz, CDCl₃) δ8.55 (d, J=1.8 Hz, 1H), 8.29 (dd, J=9.2, 1.8 Hz, 1H), 7.87 (s, 1H), 7.83 (d, J=10.2 Hz, 1H), 3.40-3.44 (m, 4H), 3.04-3.09 (m, 4H), 2.46 (s, 3H);

¹³C NMR (50 MHz, CDCl₃) δ161.5, 147.3, 141.6, 137.6, 126.5, 125.0, 122.0, 121.5, 120.5, 48.8, 44.4, 18.0;

MS (CI) m/z 273 (M⁺+1), 257, 243 (100), 173.

### EXAMPLES 2˜5

The desired compounds were prepared as the same manner in the method of Example 1 using 3-alkyl-2-chloro-6-nitroquinoline prepared in the Preparation Example 2˜5. The results of NMR analyses of the obtained compounds are set forth in the following table 2.

TABLE 2

| No | Compound |
|---|---|
| 2 | 3-ethyl-6-nitro-2-piperazin-1-yl-quinoline |
| | property: yellowish solid/yield: 66% |
| | mp130-131°C.; |
| | ¹HNMR(200MHz, CDCl₃) δ 8.60(d, J=2.6Hz, 1H), 8.30(dd, |
| | J=9.2, 2.2Hz, 1H), 7.94(s, 1H), 7.85(d, J=9.2Hz, 1H), |
| | 3.35-3.40(m, 4H), 3.04-3.09(m, 4H), 2.80(q, J=7.2Hz, |
| | 2H), 1.61(br s, 1H), 1.37(t, J=7.4Hz, 3H); |
| | ¹³CNMR(50MHz, CDCl₃) δ 161.6, 147.0, 141.6, 135.4, 131.3, |
| | 126.6, 122.4, 121.8, 120.5, 49.5, 44.4, 22.8, 12.3; |
| | MS(CI) m/z287(M⁺+1), 271, 257(100), 187. |
| 3 | 6-nitro-2-piperazin-1-yl-3-propyl-quinoline |
| | property: yellowish solid/yield: 54% |
| | mp140-141°C.; |
| | ¹HNMR(200MHz, CDCl₃) δ 8.58(d, J=2.4Hz, 1H), 8.30(dd, |
| | J=9.1, 2.5Hz, 1H), 7.92(s, 1H), 7.84(d, J=9.2Hz, 1H), |
| | 3.35-3.39(m, 4H), 3.04-3.09(m, 4H), 2.72(t, J=7.7Hz, |
| | 2H), 1.72-1.83(m, 2H), 1.67(brs, 1H), 1.01(t, J=7.3Hz, |
| | 3H); |
| | ¹³CNMR(50MHz, CDCl₃) δ 161.8, 147.1, 141.8, 136.0, 126.7, |
| | 122.3, 121.8, 120.5, 49.6, 44.4, 32.1, 21.4, 12.4; |
| | MS(CI)m/z301(M⁺+1), 285, 271(100), 201. |
| 4 | 3-(3-chloropropyl)-6-nitro-2-piperazin-1-yl-quinoline |
| | property: yellowish solid/yield: 42% |
| | mp132-133°C.; |
| | ¹HNMR(200MHz, CDCl₃) δ 8.58(d, J=2.6Hz, 1H), 8.30(dd, |
| | J=9.2, 2.6Hz, 1H), 7.93(s, 1H), 7.85(d, J=9.2Hz, 1H), |
| | 3.59(t, J=6.3Hz, 2H), 3.34-3.41(m, 4H), 3.05-3.10(m, |
| | 4H), 2.91-2.99(m, 2H), 2.17-2.24(m, 2H), 1.98(brs, 1H); |
| | ¹³CNMR(50MHz, CDCl₃) δ 161.6, 147.2, 142.0, 136.6, 128.5, |
| | 126.9, 122.3, 121.8, 120.8, 49.6, 44.3, 42.7, 30.7, 27.6. |
| 5 | 3-(3-fluoropropyl)-6-nitro-2-piperazim-1-yl-quinoline |
| | property: yellowish solid/yield: 53% |
| | mp136-138°C.; |
| | ¹HNMR(200MHz, CDCl₃) δ 8.58(d, J=2.4Hz, 1H), 8.31(dd, |
| | J=9.2, 2.6Hz, 1H), 7.93(s, 1H), 7.85(d, J=9.2Hz, 1H), |
| | 4.52(dt, J=47.4, 5.7Hz, 2H), 3.35-3.40(m, 4H), 3.05-3.10 |
| | (m, 4H), 2.92(t, J=7.9Hz, 2H), 2.32(brs, 1H), 2.13(dm, |
| | J=26.0Hz, 2H); |
| | ¹³CNMR(50MHz, CDCl₃) δ 161.6, 147.2, 141.9, 136.4, 128.8, |
| | 126.8, 122.3, 121.8, 120.8, 81.5(d, J=165Hz), 49.5, 44.2, |
| | 28.8(d, J=20Hz), 26.1. |
| | HRMS(FAB)m/z C₁₆H₁₉FN₄O₂(MH+) calcd: 319.1578; found: |
| | 319.1574. |

### EXAMPLE 6

### Preparation of 6-iodo-2-piperazin-1-yl-quinoline

Step 1) Preparation of 6-iodo-2-(4-formylpiperazin-1-yl)-quinoline

In 20 ml of DMF, 2-chloro-6-iodoquinoline (1.591 g, 5.28 mmol) prepared in the Preparation Example 6 was dissolved, added 1-piperazinecarboxaldehyde (1.922 ml, 15.84 mmol) at room temperature, and then stirred at 110° C. for 10 10 hours. The mixture was cooled, added with water to produce a precipitate, washed with 50 ml of water, and then dried over for 1 hour in vacuo to give a precipitate.

### Step 2) Preparation of 6-iodo-2-piperazin-1-yl-quinoline

In 4 M conc. H₂SO₄ aqueous solution, the product prepared in step 1) was dissolved and stirred at 100° C. for 2.5 hours. To the mixture was added 50 ml of water and further added 4 N NaOH aqueous solution to adjust pH=12. Thus obtained precipitate was isolated by filtration, washed with 50 ml of water, and then dried over for 1 hour in vacuo to give a yellowish precipitate (975 mg, 2.87 mmol, 54%).

HCl salt; ¹H NMR (D₂O) δ8.15 (d, J=10.0 Hz, 1H), 8.12 (d, J=1.8 Hz, 1H), 7.92 (dd, J=8.7, 1.9 Hz, 1H), 7.46 (d, J=8.8 Hz, 1H), 7.28 (d, J=9.6 Hz, 1H), 4.05 (t, J=5.3 Hz, 4H), 3.40 (t, J=5.3 Hz, 4H);

¹³C NMR (D₂O) δ149.9, 140.8, 139.0, 134.4, 133.0, 120.5, 117.1, 110.3, 87.6, 41.7, 40.3.

### EXAMPLE 7

### Preparation of 6-bromo-2-piperazin-1-yl-quinoline

### Step 1) Preparation of 6-bromo-2-(4-formylpiperazin-1-yl)-quinoline

In 40 ml of DMF, 2-bromo-6-iodoquinoline (1.130 g, 4.66 mmol) prepared in the Preparation Example 7 was dissolved, added 1-piperazinecarboxaldehyde (1.696 ml, 13.98 mmol) at room temperature, and then stirred at 110° C. for 5 hours. The mixture was cooled, added water to produce a solid, washed with 50 ml of water, and then dried over for 1 hour in vacuo to give a precipitate.

### Step 2) Preparation of 6-bromo-2-piperazin-1-yl-quinoline

In 30 ml of 4 M conc. H₂SO₄ aqueous solution, the product prepared in step 1) was dissolved and stirred at 100° C. for 5 hours. To the mixture was added 50 ml of water and further added 4 N NaOH aqueous solution to adjust pH=12. Thus obtained precipitate was isolated by filtration, washed with 50 ml of water, and then dried over for 1 hour in vacuo to give a yellowish precipitate (431 mg, 1.48 mmol, 32%).

HCl salt; ¹H NMR (D₂O) δ8.22 (d, J=10.0 Hz, 1H), 7.94 (d, J=2.2 Hz, 1H), 7.79 (dd, J=9.0, 2.0 Hz, 1H), 7.64 (d, J=8.8 Hz, 1H), 7.35 (d, J=9.8 Hz, 1H), 4.11 (t, J=5.1 Hz, 4H), 3.47 (t, J=5.1 Hz, 3H);

¹³C NMR (D₂O) d 150.0, 140.9, 133.6, 132.8, 128.3, 120.4, 117.5, 116.3, 110.4, 41.5, 40.3.

### EXAMPLE 8

### Preparation of 6-chloro-2-piperazin-1-yl-quinoline

### Step 1) Preparation of 6-chloro-2-(4-formylpiperazin-1-yl)-quinoline

In 20 ml of DMF, 2,6-dichloroquinoline (752 mg, 3.80 mmol) prepared in the Preparation Example 7 was dissolved, added 1-piperazinecarboxaldehyde (1.383 ml, 11.40 mmol) at room temperature, and then stirred at 110° C. for 10 hours. The mixture was cooled, added water to produce a precipitate, washed with 50 ml of water, and then dried over for 1 hour in vacuo to give the precipitate.

### Step 2) Preparation of 6-chloro-2-piperazin-1-yl-quinoline

In 30 ml of 4 M conc. H₂SO₄ aqueous solution, the product prepared in step 1) was dissolved and stirred at 100° C. for 5 hours. To the mixture was added 50 ml of water and further added 4 N NaOH aqueous solution to adjust pH=12. Thus obtained precipitate was isolated by filtration, washed with 50 ml of water, and then dried over for 1 hour in vacuo to give a yellowish precipitate (395 mg, 1.60 mmol, 42%).

HCl salt; ¹H NMR (D₂O) δ8.05 (d, J=9.6 Hz, 1H), 7.49-7.59 (m, 3H), 7.17 (d, J=9.2 Hz, 1H), 3.89 (br s, 4H), 3.26 (br s, 4H);

¹³C NMR (D₂O) d 149.6, 140.9, 132.0, 130.7, 128.4, 124.9, 119.7, 116.9, 110.1, 41.2, 39.9.

### EXAMPLE 9

### Preparation of 3-bromo-6-nitro-2-piperazin-1-yl-quinoline

### Step 1) Preparation of 3-bromo-6-nitro-2-(4-formylpiperazin-1-yl)-quinoline

In 30 ml of DMF, 3-bromo-2-chloro-6-iodoquinoline (1.0 g, 3.48 mmol) prepared in the Preparation Example 9 was dissolved, added 1-piperazinecarboxaldehyde (1.266 ml, 10.44 mmol) at room temperature, and then stirred at 130° C. for 4 hours. The mixture was cooled, added water to produce a precipitate, washed with 50 ml of water, and then dried over 5 for 1 hour in vacuo to give the precipitate.

### Step 2) Preparation of 3-bromo-6-nitro-2-piperazin-1-yl-quinoline

In 30 ml of 4 M conc. H₂SO₄ aqueous solution, the solid prepared in step 1) was dissolved and stirred at 100° C. for 5 hours. To the mixture was added 50 ml of water and further added 4 N NaOH aqueous solution to adjust pH=12. Thus obtained precipitate was isolated by filtration, washed with 50 ml of water, and then dried over for 1 hour in vacuo to give a yellowish precipitate (686 mg, 2.12 mmol, 61%).

HCl salt; ¹H NMR (D₂O) δ8.28 (s, 1H), 8.26 (s, 1H), 8.10 (d, J=9.4 Hz, 1H), 7.58 (d, J=8.8 Hz, 1H), 3.63 (br s, 4H), 3.37 (br s, 4H); ¹³C NMR (D₂O) δ156.3, 149.7, 145.2, 141.2, 125.7, 121.7, 121.2, 121.0, 110.8, 44.2, 41.5.

### EXAMPLE 10

### Preparation of 4-chloro-6-nitro-2-piperazin-1-yl-quinoline

### Step 1) Preparation of 4-chloro-6-nitro-2-(4-formylpiperazin-1-yl)quinoline

In 40 ml of DMF, 2,4-dichloro-6-nitroquinoline (263 mg, 1.08 mmol) prepared in the Preparation Example 10 was dissolved, added 1-piperazinecarboxaldehyde (0.392 ml, 3.23 mmol) at room temperature, and then stirred for 1 hour. After the completion of stirring, thus obtained mixture was added water to produce a precipitate. The precipitate was isolated by filtration, washed with 50 ml of water, and then dissolved in dichloromethane. The desired product was obtained by column chromatography affording the mixture using 5% of methanol/dichloromethane as an eluent.

### Step 2) Preparation of 4-chloro-6-nitro-2-piperazin-1-yl-quinoline

In 10 ml of 4 M conc. H₂SO₄ aqueous solution, the product prepared in step 1) was dissolved and stirred at 60° C. for 24 hours. To the mixture was added 50 ml of water and further added 4 N NaOH aqueous solution to adjust pH=12. Thus obtained precipitate was isolated by filtration, washed with 20 ml of water, and then dried over for 1 hour in vacuo to give a yellowish precipitate (158 mg, 0.54 mmol, 90%).

mp 175.2-177.0° C.;

¹H NMR (CDCl₃) δ8.89 (d, J=2.6 Hz, 1H), 8.31 (dd, J=9.5, 2.5 Hz, 1H), 7.64 (d, J=9.2 Hz, 1H), 7.14 (s, 1H), 3.79 (t, J=5.2 Hz, 4H), 3.00 (t, J=5.1 Hz, 4H);

¹³C NMR (CDCl₃) δ156.5, 150.2, 142.8, 140.7, 125.9, 122.7, 119.7, 118.0, 108.9, 44.4, 44.3;

MS (CI) m/z (relative intensity) 293 (M⁺+1, 7), 265 (29), 263 (100).

### EXAMPLE 11

### Preparation of 4-bromo-6-nitro-2-piperazin-1-yl-quinoline

In 15 ml of DMF, 2,4-dibromo-6-nitroquinoline (700 mg, 2.11 mmol) prepared in the Preparation Example 11 was dissolved, added 1-piperazinecarboxaldehyde (1.266 ml, 10.44 mmol) dissolved in 15 ml of DMF at room temperature, and then stirred for 4 hours. After the completion of stirring, thus obtained mixture was added water to produce a precipitate. Thus obtained solid was isolated by filtration, washed with 50 ml of water, and then dried over for 6 hours in vacuo to give a yellowish precipitate (508 mg, 1.39 mmol, 66%).

¹H NMR (DMSO-d₆) δ8.63 (d, J=2.6 Hz, 1H), 8.26 (dd, J=9.2, 2.6 Hz, 1H), 7.81 (s, 1H), 7.58 (d, J=9.4 Hz, 1H), 3.76 (t, J=5.0 Hz, 4H), 2.82 (t, J=5.0 Hz, 4H);

¹³C NMR (DMSO-d₆) δ156.8, 150.2, 140.4, 133.6, 126.3, 123.2, 121.8, 119.0, 114.3, 44.8, 44.6.

### EXAMPLE 12

### Preparation of 4-iodo-6-nitro-2-piperazin-1-yl-quinoline

### Step 1) Preparation of 4-iodo-6-nitro-2-(4-formylpiperazin-1-yl)quinoline

In 10 ml of ethanol, 4-tributylstannyl-6-nitro-2-(4-formylpiperazin-1-yl)quinoline (140 mg, 0.243 mmol) prepared in Preparation Example 12 was dissolved, added 1M NaI solution (0.487 ml, 0.487 mmol), 1M phosphoric acid solution (0.243 ml, 0.243 mmol) and dichloroamine T (TCI, 58 mg, 0.243 mmol), and then stirred at room temperature for 20 minutes to produce a precipitate. The solvent was evaporated over under vacuum to give the desired compound, a solid.

### Step 2) Preparation of 4-iodo-6-nitro-2-piperazin-1-yl-quinoline

In 10 ml of 4 M conc. H₂SO₄ aqueous solution, the product prepared in step 1) was dissolved and stirred at 80° C. for 1 hour. To the mixture was added 15 ml of water and further added 4 N NaOH aqueous solution to adjust pH=12. Thus obtained precipitate was isolated by filtration, washed with 50 ml of water, and then dried over for 3 hours in vacuo to give a yellowish precipitate (86 mg, 93%).

¹H NMR (DMSO-d₆) δ8.55 (d, J=2.2 Hz, 1H), 8.20 (dd, J=9.2, 2.6 Hz, 1H), 7.96 (s, 1H), 7.51 (d, J=9.2 Hz, 1H), 3.82 (t, J=4.8 Hz, 4H), 2.77 (t, J=4.6 Hz, 4H);

¹³C NMR (DMSO-d₆) δ156.4, 149.0, 141.1, 126.9, 126.6, 123.2, 122.1, 121.6, 113.6, 42.1, 41.3.

### EXAMPLE 13

### Preparation of 6-nitro-2-piperazin-1-yl-4-vinylquinoline

In 5 ml of dioxane, 4-bromo-6-nitro-2-(4-formylpiperazin-1-yl)quinoline (200 mg, 0.5477 mmol) prepared in step 1) of Preparation Example 12 and tetrakis(triphenylphosphine)palladium (0) (15 mg, 0.6024 mmol) were dissolved, added tributyl(vinyl)tin (0.181 ml, 0.6024 mmol) at room temperature, and then refluxed at 100° C. for 2.5 hours. After the completion of the reaction, the mixture was cooled to room temperature, filtered using celite, thus obtained filtrate was added a mixture of water and ammonia water, and then extracted with dichloromethane. The desired product as a yellowish solid was obtained by column chromatography affording the mixture (130 mg, 84%).

¹H NMR (CDCl₃) δ8.77 (d, J=2.2 Hz, 1H), 8.29 (dd, J=9.1, 2.5 Hz, 1H), 7.66 (d, J=9.2 Hz, 1H), 7.28 (dd, J=17.2, 11.0 Hz, 1H), 7.06 (s, 1H), 5.91 (d, J=17.2 Hz, 1H), 5.68 (d, J=11.0 Hz, 1H), 3.84 (t, J=4.9 Hz, 4H), 3.02 (t, J=4.9 Hz, 4H);

¹³C NMR (CDCl₃) δ157.0, 150.2, 144.9, 130.6, 125.9, 122.2, 121.9, 119.7, 119.3, 118.2, 105.4, 44.4, 44.2.

### EXAMPLE 14

### Preparation of 4-(2-furanyl)-6-nitro-2-piperazin-1-yl-quinoline

### Step 1) Preparation of 4-(2-furanyl)-6-nitro-2-(4-formyl-piperazin-1-yl)quinoline

In 7 ml of dioxane, 4-bromo-6-nitro-2-(4-formylpiperazin-1-yl)quinoline (300 mg, 0.82 mmol) prepared in step 1) of Preparation Example 12, CuI (15 mg, 0.08 mmol) and tetrakis(triphenylphosphine)Palladium (0) (19 mg, 0.02 mmol) was dissolved, added tributyl(2-furanyl)tin (587 mg, 1.64 mmol) at room temperature, and then refluxed for 24 hours. After the completion of refluxing, the mixture was cooled to room temperature, added 10 ml of 1% KF solution, and then stirred at room temperature for 10 hours to produce a precipitate. The precipitate was filtered off, the remained filtrate was add 10 ml of water and extracted with ethylacetate. The desired product was obtained by column chromatography affording the mixture.

### Step 2) Preparation of 4-(2-furanyl)-6-nitro-2-piperazin-1-yl-quinoline

In 10 ml of 4 M conc. H₂SO₄ aqueous solution, the compound (4-(2-furanyl)-6-nitro-2-(4-formylpiperazin-1-yl) prepared in step 1) was dissolved and stirred at 70° C. for 10 hours. To the mixture was added 15 ml of water and further added 4 N NaOH aqueous solution to adjust pH=12. Thus obtained precipitate was isolated by filtration, washed with 50 ml of water, and then dried over for 3 hours in vacuo to give a yellowish precipitate (206 mg, 77%).

¹H NMR (CDCl₃) δ9.15 (d, J=2.6 Hz, 1H), 8.31 (dd, J=9.1, 2.5 Hz, 1H), 7.69 (d, J=2.0 Hz, 1H), 7.69 (d, J=9.2 Hz, 1H), 7.25 (s, 1H), 6.98 (d, J=3.4 Hz, 1H), 6.65 (dd, J=3.6, 1.6 Hz, 1H), 3.86 (t, J=4.9 Hz, 4H), 3.03 (t, J=5.1 Hz, 4H), 1.71 (br s, 1H);

¹³C NMR (CDCl₃) δ155.9, 150.0, 147.8, 141.7, 140.0, 136.0, 125.3, 121.1, 120.2, 112.2, 109.8, 109.7, 105.4, 43.7, 43.5.

### EXAMPLE 15

### Preparation of 2-(3-methylpiperazin-1-yl)-6-nitro-quinoline

In 10 ml of DMF, 2-chloro-6-nitroquinoline (500 mg, 2.40 mmol) prepared in Preparation Example 13 was dissolved, added 2-methylpiperazine (721 mg, 7.20 mmol) at room temperature, and then reacted at 70° C. for 3 hours. After the completion of the reaction, the mixture was cooled to room temperature, added water to produce a precipitate. The solid was isolated by filtration, washed with 50 ml of water, and then dried over for 3 hours in vacuo to give the desired product as a yellowish solid (556 mg, 2.04 mmol, 85%).

¹H NMR (CDCl₃) δ8.42 (d, J=2.6 Hz, 1H), 8.20 (dd, J=9.3, 1.9 Hz, 1H), 7.86 (d, J=9.4 Hz, 1H), 7.57 (d, J=9.0 Hz, 1H), 6.99 (d, J=9.4 Hz, 1H), 4.45 (d, J=11.2 Hz, 1H), 2.63-3.14 (m, 5H), 1.75 (br s, 1H), 1.35 (d, J=6.0 Hz, 3H);

¹³C NMR (CDCl₃) δ156.81, 149.9, 140.1, 136.9, 125.3, 122.6, 121.9, 119.3, 109.2, 50.2, 48.9, 44.1, 43.4, 17.9.

### EXAMPLE 16

### Preparation of 2-(N-methyl-N-(2-N′-methylamino)ethyl)amino-6-nitro-quinoline

In 10 ml of DMF, 2-chloro-6-nitroquinoline (500 mg, 2.40 mmol) was dissolved, added N,N′-dimethylethan-1,2-diamine (635 mg, 7.20 mmol) at room temperature, and then reacted at 100° C. for 5 hours. After the completion of the reaction, the mixture was cooled to room temperature, added water to produce a precipitate. The precipitate was isolated by filtration, washed with 50 ml of water, and then dried over for 3 hours in vacuo to give the desired product as a yellowish solid (487 mg, 1.87 mmol, 78%).

HCl salt; ¹H NMR (D₂O) δ8.66 (d, J=2.4 Hz, 1H), 8.33-8.42 (m, 2H), 7.91 (d, J=9.2 Hz, 1H), 7.34 (d, J=8.0 Hz, 1H), 4.08 (t, J=6.9 Hz, 2H), 3.24-3.38 (m, 5H), 2.68 (s, 3H).

¹³C NMR (D₂O) δ153.2, 143.5, 143.2, 139.5, 126.2, 124.3, 119.9, 118.6, 112.6, 47.9, 44.6, 38.1, 32.7.

### EXAMPLE 17

### Preparation of 2-[1,4]diazepin-1-yl-6-nitro-quinoline

In 10 ml of DMF, 2-chloro-6-nitroquinoline (500 mg, 2.40 mmol) was dissolved, added diazepine (894 mg, 7.20 mmol) at room temperature, and then reacted at 80° C. for 5 hours. After the completion of the reaction, the mixture was cooled to room temperature, added water to produce a precipitate. The precipitate was isolated by filtration, washed with 50 ml of water, and then dried over for 3 hours in vacuo to give the desired product as a yellowish solid (602 mg, 2.21 mmol, 92%).

¹H NMR (CDCl₃) δ8.52 (d, J=2.6 Hz, 1H), 8.27 (dd, J=9.3, 2.7 Hz, 1H), 7.93 (d, J=9.2 Hz, 1H), 7.62 (d, 9.4 Hz, 1H), 6.97 (d, J=9.0 Hz, 1H), 3.87-3.93 (m, 4H), 3.11 (t, J=5.3 Hz, 2H), 2.89 (t, J=5.9 Hz, 2H), 2.22 (br s, 1H), 1.91-2.03 (m, 2H);

¹³C NMR (DMSO-d₆) δ157.1, 150.5, 139.4, 137.7, 125.4, 123.6, 122.2, 119.7, 46.92, 46.03, 45.41, 37.2, 27.5.

### FORMULATION EXAMPLE 1

### Formulation of a Syrup

A formulation of syrup containing the compound of the present invention as an effective ingredient in an amount of 2 wt % is described as follows.

In 80 g of warm water, the compound of the invention, saccharin, and sugar were dissolved, cooled, added with glycerin, saccharin, ointments, ethanol, sorbic acid, and distilled water. Water was added to the thus obtained mixture to be adjusted to 100 ml of total volumn. The above-mentioned acid salt can be preferably replaced with another salt.

| Ingredients | |
|---|---|
| 4-chloro-6-nitro-2-piperazin-1-yl-quinoline HCl | 2 g |
| saccharin | 0.8 g |
| sugar | 25.4 g |
| glycerin | 8.0 g |
| ointment | 0.04 g |
| ehanol | 4.0 g |
| sorbic acid | 0.4 g |
| distilled water | balance |

### FORMULATION EXAMPLE 2

### Formulation of a Tablet

A tablet containing 15 mg of effective ingredient is made as follows.

After 4-chloro-6-nitro-2-piperazin-1-yl-quinoline HCl was combined with lactose, starch and colloidal silicate, 10% gelatin solution was added to the mixture. Then, the mixture was ground, passed through a sieve with 14 mesh, dried and added with starch, talc, magnesium stearate. The tablet was obtained by injection pressing using the mixture.

| Ingredients | |
|---|---|
| 4-chloro-6-nitro-2-piperazin-1-yl-quinoline HCl | 250 g |
| lactose | 175.9 g |
| starch | 180 g |
| colloidal silicate | 32 g |
| 10% gelatin solution | q.c |
| starch | 160 g |
| talc | 50 g |
| magnesium stearate | 5 g |

### FORMULATION EXAMPLE 3

### Formulation of an Injection

An injection containing 10 mg of effective ingredient is made as follows.

After 4-chloro-6-nitro-2-piperazin-1-yl-quinoline HCl, NaCl, and ascorbic acid was dissolved in distilled water, an ampule was injected, and then sterilized.

| Ingredients | |
|---|---|
| 4-chloro-6-nitro-2-piperazin-1-yl-quinoline HCl | 1 g |
| NaCl | 0.6 g |
| ascorbic acid | 0.1 g |
| distilled water | balance |

### PREPARATION EXAMPLE 14

### Preparation of a Synaptic Membrane

After male SD Rats (250˜350 g) were decapitate by using quillotine, the whole brains were removed. The cerebral cortex were carefully cut to slices and weighed. 0.32 M sucrose which is 10 times of sucrose was added to the slices, and then homogenized twice for 15 seconds using Polytron. After being centrifuged at 4° C. for 10 minutes in 1,000×g, the thus obtained supernatant was re-centrifuged at 20,000×g for 20 minutes and then discarded. The precipitate was isolated, re-suspended in deionized water, was provided with osmotic shock for 10 minutes, and then centrifuged at 80,000×g for 20 minutes. The thus obtained precipitate was suspended in 50 ml of triacetate buffer, re-centrifuged at 48,000×g, and then transferred to a refrigerator. On the day of assay, the cells were thawed on ice, washed by centrifugation (48,000×g, 20 minutes). The obtained synaptic membrane showed 1.5 mg/ml of protein.

### EXPERIMENTAL EXAMPLE 1

### Radioligand Binding Assay

To determine the activity of the compounds according to the present invention, the sertonin binding assay was carried out.

In this example, 1 nM of [³H]citalopram was used as the radioligand. To 50 mM of tris HCl buffer the radioligand, synaptic membrane prepared in Preparation Example 14, and the compounds of the invention (200 μL) were added till final amount reached 0.6 mL. The mixture was shaken at 25° C. for 60 minutes, incubated, filtered using a Brandel Cell Harvester through a GF/B Watman filter presoaked in 0.05 polyethylimine. The filters were then washed twice with 5 ml of ice-cold buffer, dried, added with 7 ml of ultima gold scintillation cocktail, and then shaken for 3˜4 hours. The radioactivity of the filters was determined using the liquid scintillation counter. 30 μM of Fluoxetin was used to define a non-specific binding, and the concentration of [³H]citalopram in the range of 0.01˜30 nM went through 15 different experiments to define a saturation binding.

The Ki value showing the serotonin binding activity of compound of the invention was calculated by the following calculation formula 1. In this case, [F] means the concentration of free radioligand, and Kd is the affinity of radioligand with serotonin. As a result of determining an equilibrium-saturation experiment by a scatchard analysis, the Kd in this experiment was 1.12 nM. The thus obtained value of IC50 and Kd was calculated through a prism computer program and the results are as follows in Table 3.

$$Ki = \frac{{IC}_{50}}{( 1 + [ F ] / Kd )} \qquad Calculation formula 1$$

TABLE 3

| compound | Ki (Nm) |
|---|---|
| Exmaple 1 | 8.45 ± 0.62 |
| Exmaple 2 | 0.36 ± 0.02 |
| Exmaple 3 | 0.26 ± 0.01 |
| Exmaple 4 | 1.08 ± 0.17 |
| Exmaple 5 | 0.32 ± 0.01 |
| Exmaple 6 | 6.60 ± 0.52 |
| Exmaple 7 | 0.91 ± 0.07 |
| Exmaple 8 | 1.68 ± 0.13 |
| Exmaple 9 | 12.62 ± 1.14 |
| Exmaple 10 | 0.017 ± 0.05 |
| Exmaple 11 | — |
| Exmaple 12 | — |
| Exmaple 13 | 1.42 |
| Exmaple 14 | 6.4 |
| Exmaple 15 | 22.19 ± 1.70 |
| Exmaple 16 | 8.43 ± 0.45 |
| Exmaple 17 | 1.90 ± 0.15 |
| Fluoxetine | 22.13 ± 1.77 |
| Paroxetin | 0.53 ± 0.08 |
| 6-nitro-2- | 0.17 ± 0.03 |
| piperazin-1-yl- | |
| quinoline | |

As see from the Table 3, the compounds of the invention show a significantly greater affinity (see the Ki value) than the most popular Fluoxetine, especially; the compounds such as 3-ethyl-6-nitro-2-piperazin-1-yl-quinoline (Example 2), 6-nitro-2-pipearazin-1-yl-3-propylquinoline (Example 3), and 3-(3-fluoropropyl)-6-nitro-2-piperazin-1-yl-quinoline (Example 5) exceed that of Fluoxetine. In addition, having a very low Ki value of 0.017 nM, which is the most superior among any known compounds, the 4-chloro-6-nitro-2-piperazin-1-yl-quinoline in Example 10 shows an activity 10 times higher than that of 6-nitro-2-piperazin-1-yl-quinoline.

### EXPERIMENT EXAMPLE 2

### Assay for Anti-Depression Activity using a Forced Swimming Test

To determine the activity of the compounds according to the present invention, the forced swimming test was carried out.

After 4-chloro-6-nitro-2-piperazin-1-yl-quinoline was dissolved in dimethylsulfoxide in a variety of concentrations and injected to depression-induced mice, the time of immobility was measured. The immobility time may be evaluated by measuring the effect against anti-depression. Because immobility means a helplessness of the mice, in this case, slow immobility should be understood to reduce depression.

The mice were kept in a cage for 10-15 minutes wherein their feet did not reach the bottom, and then removed from the cage to induce depression. 4-chloro-6-nitro-2-piperazin-1-yl-quinoline was orally administered after 23 hours from the first treatment, subsequently, after 24 hours, the animals were placed in the cage to carry out the second treatment and the immobility time was measured for 5 minutes. The immobility caused by the second treatment was recorded while watching video tape. In order to make an objective observation, we made an average of the results of make than 3 people.

As a result, the group administered with 4-chloro-6-nitro-2-piperazin-1-yl-quinoline showed an anti-depression effect 30% higher than that of the control (p<0.003). Accordingly, this result is an evidence that the present compounds has an anti-depression effect, and thus, should be useful in treating serotonin-related disorder.

### EXPERIMENTAL EXAMPLE 3

### Assay for Anti-Depression Activity using a Tail Suspension Test

The inventors carried out the tail suspension test to evaluate an anti-depression activity of the present compound. ICR mice were divided into 5 groups made up of 8 subjects, and orally dosed with 4-chloro-6-nitro-2-piperazin-1-yl-quinoline dissolved in saline buffer solution in a variety of its concentration 60 minutes prior to the beginning of the experiment. The evaluation of the activity was estimated using administering group, negative, and positive group. After suspending the animal by the tail induced the immobility, the immobility time was measured 6 minutes watching a recorded tape for video. The final data was obtained an average value measured by 3 persons to accurate the data.

As a result, 4-chloro-6-nitro-2-piperazin-1-yl-quinoline shows an anti-depression effect 60% higher than that of control, when 50 mg/kg was administered, and anti-depression effect 56% higher than that of the control, when 5 mg/kg was administered. In addition, FIG. 2B shows that the present compounds have an effect about 65% higher than that of the control (anova, p<0.005). Because there is a task showing better effects the drug, it may show a more superior effect when 1 mg/kg is administered.

This result shows that 4-chloro-6-nitro-2-piperazin-1-yl-quinoline of the present invention effective against depression. Especially, according to the evenness shown in this test, wherein the effectiveness obtained from 1 to 50 mg/kg is almost alike, ED₅₀ of 4-chloro-6-nitro-2-piperazin-1-yl-quinoline is expected to be below 1 mg/kg.

### EXPERIMENTAL EXAMPLE 4

### Acute Toxicity in Rats Tested via Oral Administration

The following experiment was performed to see if the compounds of formula 1 have acute toxicity in rats.

6-week old SPF SD line rats were used in the tests for acute toxicity. 6-chloro-2-piperazin-1-yl-quinoline or 4-chloro-6-nitro-2-piperazin-1-yl-quinoline were suspended in 1 ml saline buffer solution and patentially administered to anterior tibialis once to 6 rats per group at the dosage of 500 mg/kg. Death, clinical symptoms, and weight change in rats were observed, hematological tests and biochemical tests of blood performed, and any abnormal signs in the gastrointestinal organs of chest and abdomen checked with eyes during autopsy. The results showed that the test compounds did not cause any specific clinical symptoms, weight change, or death in rats. No change was observed in hematological tests, biochemical tests of blood, and autopsy. The compounds used in this experiment are evaluated to be safe substances since they do not cause any toxic changes in rats up to the level of 500 mg/kg and their estimated LD₅₀ values are much greater than 500 mg/kg in rats.

Industrial Applicability

As described hereinbefore, the quinoline compounds represented by formula 1 show an excellent binding affinity with serotonin transporter, show an excellent anti-depression activity by measuring immobility time using the forced swimming test and tail suspension test. Accordingly, the present compounds should be useful in treating and preventing serotonin-related mental disorders. In addition, the method according to the present invention has also an advantage of readily preparing the compounds of the formula 1.

1. A quinoline derivative of formula 1, or pharmaceutically acceptable salt of the same: 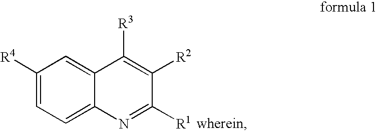
R¹ is piperazinyl, 2-methylpiperazinyl, perhydrodiazepinyl or N-methyl-N-(2-N′-methylamino)ethylamine group;
R² is H, halogen atom, C₁˜C₄ alkyl or C₁˜C₄ haloalkyl;
R³ is H, halogen atom, vinyl or furanyl group; and
R⁴ is halogen atom or nitro group
with the proviso that
when R¹ is piperazinyl or 2-methylpiperazinyl;
R³ is H; and
R⁴ is nitro group,
R² is not H, halogen atom, C₁˜C₃ alkyl, or C₁˜C₂ haloalkyl.

2. The derivative of claim 1, wherein R¹ is 2-methylpiperazinyl, perhydrodiazepinyl or N-methyl-N-(2-N′-methylamino)ethylamine;
R² is H, bromine, methyl, ethyl, chloropropyl or fluoropropyl;
R³ is H, chlorine, bromine, iodine, vinyl or 2-furanyl group; and
R⁴ is chlorine, bromine, iodine, or nitro group
with the proviso that
when R¹ is 2-methylpiperazinyl;
R³ is H; and
R⁴ is nitro group,
R² is not H, bromine, methyl or ethyl.

3. The derivative of claim 1, wherein the derivative is selected from the group consisting of:
3-(3-chloropropyl)-6-nitro-2-piperazin-1-yl-quinoline;
3-(3-fluoropropyl)-6-nitro-2-piperazin-1-yl-quinoline;
6-iodo-2-pipeerazin-1-yl-quinoline;
6-bromo-2-piperazine-1-yl-quinoline;
6-chloro-2-piperazin-1-yl-quinoline;
4-chloro-6-nitro-2-piperazin-1-yl-quinoline;
4-bromo-6-nitro-2-piperazin-1-yl-quinoline;
4-iodo-6-nitro-2-piperazin-1-yl-quinoline;
6-nitro-2-piperazin-1-yl4-vinylquinoline;
4-(2-furanyl)-6-nitro-2-piperazin-1-yl-quinoline;
2-(N-methyl-N-(2-N′-methylamino)ethyl)amino-6-nitroquinoline; and
2-perhydrodiazepin-1-yl-6-nitoquinoline.

4. A method for preparing a compound of formula 3, which comprises substituting a quinoline compound of formula 2 with piperazine to introduce a piperazinyl group at 2-position of the quinoline compound of formula 2: 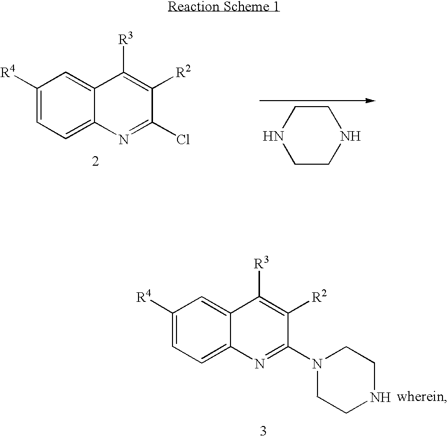
R² is H, halogen atom, C₁˜C₄ alkyl or C₁˜C₄ haloalkyl;
R³ is H, halogen atom, vinyl or furanyl group; and
R⁴ is halogen atom or nitro group.

5. A pharmaceutical composition comprising the quinoline derivative of the formula 1 as an effective ingredient for preventing or treating serotonin-related mental disorder.

6. The composition of claim 1, wherein the mental disorder is a depression.
